(19) United States
(12) Patent Application Publication
(10) Pub. No.: US 2025/0345633 A1
(43) Pub. Date: Nov. 13, 2025

(54) COMPUTER-IMPLEMENTED METHOD FOR USE IN A RADIOTHERAPY WORKFLOW

(71) Applicant: Elekta, Inc., Atlanta, GA (US)

(72) Inventors: Elizabeth Jean Lansing, Atlanta, GA (US); Michael McGrain Flores, Atlanta, GA (US); Michael Meltsner, Atlanta, GA (US); Todd Joseph Wilfling, St. Louis, MO (US)

(21) Appl. No.: 18/661,025

(22) Filed: May 10, 2024

(51) Int. Cl. A61N 5/10; G16H 40/63

(52) U.S. Cl. CPC A61N 5/107; A61N 5/1039; G16H 40/63; A61N 2005/1055; A61N 2005/1074

(57) ABSTRACT

A computer-implemented method for use in a radiotherapy workflow, comprises loading a reference image and obtaining at least one localization image of a patient positioned on an adjustable patient support. The method can further comprise loading the at least one localization image for display for user image quality review and for comparison with the reference image to identify patient support adjustment values and permit user image registration optimization. The at least one localization image and the reference image are loaded for display at a common display location, the patient support adjustment values are displayed for patient setup positioning error review, and upon receiving user input confirming the at least one localization image is of acceptable quality and image registration is optimized, the patient support adjustment values are sent for application to the adjustable patient support.

This disclosure relates to radiotherapy workflows, and in particular to a system and method for use in a radiotherapy workflow.

### BACKGROUND

Radiotherapy can be described as the use of ionising radiation, such as X-rays, to treat a human or animal body. Radiotherapy is commonly used to treat tumours within the body of a patient or subject. In such treatments, ionising radiation is used to irradiate, and thus destroy or damage, cells which form part of the tumour. In radiotherapy treatment, it is desirable to deliver a prescribed dose of radiation to a target region, e.g. a tumour, of a patient and to limit irradiation of other parts of the patient, e.g. healthy tissue and organs at risk (OARs).

The treatment planning procedure typically involves obtaining one or more medical images, such as a CT image of the patient, and segmenting them to identify the target region and OARs near the target region. The segmentation process can be performed manually or using auto-segmentation techniques. The clinician determines radiation treatment parameters, for example by prescribing a radiation dose to be delivered to the target region and maximum doses which can safely be delivered to the various OARs. The treatment planning procedure may then involve optimizing various radiation delivery variables to meet the prescribed radiation treatment parameters, for example determining the number of sessions (or ‘fractions’) over which radiotherapy should be conducted, the angles at which the radiation beam should be applied during each fraction, at what beam energy, the duration of application of the radiation beams at these angles, and the beam shape(s) at each angle of delivery. The clinician can be assisted by software during some or all of these steps. This aspect of treatment planning, conducted in advance of the patient's treatment (and sometimes between treatment fractions if there are gradual changes, making the need for a plan adaptation foreseeable), can be described as “offline” treatment planning as part of an offline adaptive treatment workflow.

However, the characteristics of the tumour, such as its shape and size, may change over the course of the multiple fractions of treatment. Similarly, other patient information and characteristics of the patient's anatomy can change between fractions, in both cases in a less foreseeable way. Accordingly, “online” adaptive radiotherapy techniques may be used to update and re-optimise the radiotherapy treatment plan and/or delivery variables immediately prior to the patient's treatment. According to online adaptive radiotherapy techniques, the patient is imaged again immediately prior to treatment, and the treatment plan for that day's fraction may be adjusted and/or reoptimized according to the latest available medical images. Online adaptive radiation therapy techniques, as part of an online treatment workflow, therefore allow inter-fraction anatomical changes to be taken into account.

Currently available online adaptive treatment workflows require a lot of manual steps and decisions, each of which require appropriate skills and clinical experience at the point of treatment delivery. In other words, current online adaptive workflows require a high level of expertise and experience to use effectively, and experienced clinicians must be present on-site for the workflow to operate, which is time and resource intensive. In addition, currently available treatment workflows require upfront decisions by clinicians on the appropriate care path for the patient. In other words, clinicians must decide before the start of treatment if a patient will experience the ‘standard’ (or ‘traditional’) IGRT workflow, or the online adaptive workflow.

### BRIEF DESCRIPTION OF THE FIGURES

Specific examples are now described, by way of example only, with reference to the drawings, in which:

FIG. 1 shows a radiotherapy device or apparatus according to the present disclosure;

FIG. 2 shows a radiotherapy treatment workflow according to the present disclosure;

FIG. 3 shows a computer-implemented method for use in a radiotherapy workflow according to the present disclosure;

FIG. 4 shows a computer implemented method for use in a radiotherapy workflow according to the present disclosure in further detail;

FIG. 5a shows a localisation image display;

FIG. 5b shows a reference image display;

FIG. 6 shows an interrupt workflow recommendation screen;

FIG. 7 shows a fused image display;

FIG. 8 shows a block diagram of one implementation of a radiotherapy system; and

FIG. 9 shows a computer readable medium or, more generally, a computer program product.

### SUMMARY

Placement of the patient relevant to the radiation treatment beam is an important step during the radiation therapy treatment session, to ensure correct positioning of the patient for subsequent treatment. In known approaches this is done by acquiring 2D and/or 3D localisation/localization images by any appropriate means such as CBCT or portal imaging, of the patient at their initial position on the treatment table. In some examples, such arrangements require a multi-step decision making and re-imaging process involving both assessment of whether the localisation images are fit for use (meaning that the image quality is sufficient for image registration and evaluation purposes) and ensuring the that patient set up is executed satisfactorily. In either event, if the criteria is not met, then this can involve repositioning of the patient, acquisition of a new set of images and/or re-imaging, forcing serial decision making.

In an example of the present disclosure, to address the issues discussed above, a reference image of a patient and optionally a plan, which indicates the desired position of the patient relative to the radiation beam, is loaded, and a localisation/localization image of a patient positioned on an adjustable patient support, for example the patient support surface, is obtained and loaded for display. This permits both user image quality review and comparison of the localisation image with the reference image and user image registration optimization by loading them for display at a common display location together with display of the patient support adjustment values for patient setup positioning error review.

As a result, when user input is received confirming the image is of acceptable quality and image registration is optimized, the patient support adjustment values determined by the comparison can be sent for application to the patient support. Accordingly, the image review process and follow-on workflow can be streamlined and, once the patient set-up and image quality criteria have been met, the workflow can be continued by comparing and editing if needed the position of the localisation image relative to the reference plan images to provide correction values needed to fine-tune the table position. The corrections can then be sent to the system responsible for moving the table so that the patient is aligned to the beam as intended by the reference plan, for example upon acceptance by the user. By providing a blended or fused display, image assessment and reference can be provided in a more efficient way and in particular data and display reload can be avoided.

### DETAILED DESCRIPTION

Disclosed herein are systems, devices, methods and apparatuses relating to radiotherapy. With linear accelerator-based radiotherapy devices being highly complex and having many inter-related parts, the terms “system”, “device”, “apparatus”, and “machine” may all be applied interchangeably to describe the radiotherapy device as a whole, or collections of components of the radiotherapy device.

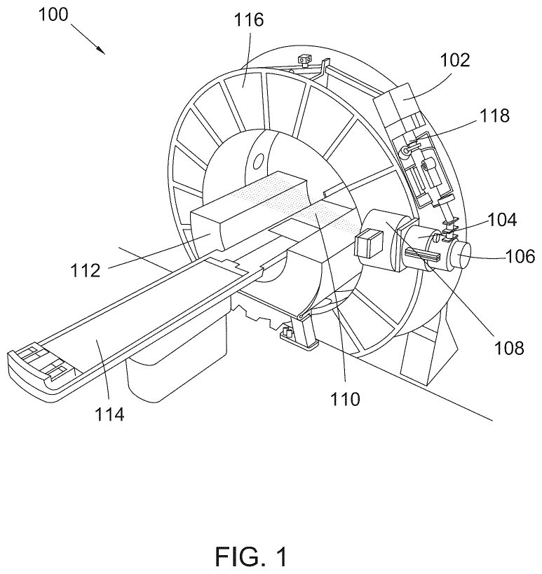

FIG. 1 shows an exemplary radiotherapy (RT) device 100. The device and its constituent components will be well known to the skilled person but is described here generally for the purpose of providing useful accompanying information for the present disclosure. The radiotherapy device 100 is based on a linear accelerator (linac).

The device shown in FIG. 1 combines magnetic resonance (MR) imaging capability with a linac-based radiotherapy capability, and is known as an MR-linac device. MR-linacs are particularly well-suited for delivery of adaptive treatment, since MR images may be taken immediately prior to or during treatment. The device and its constituent components will be described generally for the purpose of providing useful accompanying information for the present disclosure. The device depicted in FIG. 1 is in accordance with the present disclosure and is suitable for use with the disclosed systems and apparatuses. However, the present disclosure may be implemented in any radiotherapy device, for example, a linac-based radiotherapy device with CBCT imaging capability.

The device 100 in FIG. 1 comprises both MR imaging apparatus 112 and radiotherapy (RT) apparatus which may comprise a linac device. The MR imaging apparatus 112 is shown in cross-section in the diagram. In operation, the MR scanner produces MR images of the patient, and the linac device produces and shapes a beam of radiation and directs it toward a target region within a patient's body in accordance with a radiotherapy treatment plan. The depicted device does not have the usual ‘housing’ which would cover the MR imaging apparatus 112 and RT apparatus in a commercial setting such as a hospital.

The MR-linac device depicted in FIG. 1 comprises a source of radiofrequency waves 102, a waveguide 104, a source of electrons 106, a source of radiation 106, a collimator 108 such as a multi-leaf collimator configured to collimate and shape the beam, MR imaging apparatus 112, and a patient support surface 114. In use, the device would also comprise a housing (not shown) which, together with the ring-shaped gantry, defines a bore. The moveable support surface 114 can be used to move a patient, or other subject, into the bore when an MR scan and/or when radiotherapy is to commence. The MR imaging apparatus 112, RT apparatus, and a subject support surface actuator are communicatively coupled to a controller or processor. The controller is also communicatively coupled to a memory device comprising computer-executable instructions which may be executed by the controller.

The RT apparatus comprises a source of radiation and a radiation detector (not shown). Typically, the radiation detector is positioned diametrically opposed to the radiation source. The radiation detector is suitable for, and configured to, produce radiation intensity data. In particular, the radiation detector is positioned and configured to detect the intensity of radiation which has passed through the subject. The radiation detector may also be described as radiation detecting means, and may form part of a portal imaging system.

The radiation source may comprise a beam generation system. For a linac, the beam generation system may comprise a source of RF energy 102, an electron gun 106, and a waveguide 104. The radiation source is attached to the rotatable gantry 116 so as to rotate with the gantry 116. In this way, the radiation source is rotatable around the patient so that the treatment beam 110 can be applied from different angles around the gantry 116. In a preferred implementation, the gantry is continuously rotatable. In other words, the gantry can be rotated by 360 degrees around the patient, and in fact can continue to be rotated past 360 degrees. The gantry may be ring-shaped. In other words, the gantry may be a ring-gantry.

The source 102 of radiofrequency waves, such as a magnetron, is configured to produce radiofrequency waves. The source 102 of radiofrequency waves is coupled to the waveguide 104 via circulator 118, and is configured to pulse radiofrequency waves into the waveguide 104. Radiofrequency waves may pass from the source 102 of radiofrequency waves through an RF input window and into an RF input connecting pipe or tube. A source of electrons 106, such as an electron gun, is also coupled to the waveguide 104 and is configured to inject electrons into the waveguide 104. In the electron gun 106, electrons are thermionically emitted from a cathode filament as the filament is heated. The temperature of the filament controls the number of electrons injected. The injection of electrons into the waveguide 104 is synchronised with the pumping of the radiofrequency waves into the waveguide 104. The design and operation of the radiofrequency wave source 102, electron source and the waveguide 104 is such that the radiofrequency waves accelerate the electrons to very high energies as the electrons propagate through the waveguide 104.

The design of the waveguide 104 depends on whether the linac accelerates the electrons using a standing wave or travelling wave, though the waveguide typically comprises a series of cells or cavities, each cavity connected by a hole or ‘iris’ through which the electron beam may pass. The cavities are coupled in order that a suitable electric field pattern is produced which accelerates electrons propagating through the waveguide 104. As the electrons are accelerated in the waveguide 104, the electron beam path is controlled by a suitable arrangement of steering magnets, or steering coils, which surround the waveguide 104. The arrangement of steering magnets may comprise, for example, two sets of quadrupole magnets.

Once the electrons have been accelerated, they may pass into a flight tube. The flight tube may be connected to the waveguide by a connecting tube. This connecting tube or connecting structure may be called a drift tube. The electrons travel toward a heavy metal target which may comprise, for example, tungsten. Whilst the electrons travel through the flight tube, an arrangement of focusing magnets act to direct and focus the beam on the target.

To ensure that propagation of the electrons is not impeded as the electron beam travels toward the target, the waveguide 104 is evacuated using a vacuum system comprising a vacuum pump or an arrangement of vacuum pumps. The pump system is capable of producing ultra-high vacuum (UHV) conditions in the waveguide 104 and in the flight tube. The vacuum system also ensures UHV conditions in the electron gun. Electrons can be accelerated to speeds approaching the speed of light in the evacuated waveguide 104.

The source of radiation is configured to direct a beam 110 of therapeutic radiation toward a patient positioned on the patient support surface 114. The source of radiation may comprise a heavy metal target toward which the high energy electrons exiting the waveguide are directed. When the electrons strike the target, X-rays are produced in a variety of directions. A primary collimator may block X-rays travelling in certain directions and pass only forward travelling X-rays to produce a treatment beam 110. The X-rays may be filtered and may pass through one or more ion chambers for dose measuring. The beam can be shaped in various ways by beam-shaping apparatus, for example by using a multi-leaf collimator 108, before it passes into the patient as part of radiotherapy treatment.

In some implementations, the source of radiation is configured to emit either an X-ray beam or an electron particle beam. Such implementations allow the device to provide electron beam therapy, i.e. a type of external beam therapy where electrons, rather than X-rays, are directed toward the target region. It is possible to ‘swap’ between a first mode in which X-rays are emitted and a second mode in which electrons are emitted by adjusting the components of the linac. In essence, it is possible to swap between the first and second mode by moving the heavy metal target in or out of the electron beam path and replacing it with a so-called ‘electron window’. The electron window is substantially transparent to electrons and allows electrons to exit the flight tube.

The subject or patient support surface 114 is configured to move between a first position substantially outside the bore, and a second position substantially inside the bore. In the first position, a patient or subject can mount the patient support surface. The support surface 114, and patient, can then be moved inside the bore, to the second position, in order for the patient to be imaged by the MR imaging apparatus 112 and/or imaged or treated using the RT apparatus. The movement of the patient support surface is effected and controlled by a subject support surface actuator, which may be described as an actuation mechanism. The actuation mechanism is configured to move the subject support surface in a direction parallel to, and defined by, the central axis of the bore. The terms subject and patient are used interchangeably herein such that the subject support surface can also be described as a patient support surface. The subject support surface may also be referred to as a moveable or adjustable couch or table.

The radiotherapy apparatus/device depicted in FIG. 1 also comprises MR imaging apparatus 112. The MR imaging apparatus 112 is configured to obtain images of a patient or subject positioned, i.e. located, on the subject support surface 114. The MR imaging apparatus 112 may also be referred to as the MR imager. The MR imaging apparatus 112 may be a conventional MR imaging apparatus operating in a known manner to obtain MR data, for example MR images. The skilled person will appreciate that such a MR imaging apparatus 112 may comprise a primary magnet, one or more gradient coils, one or more receive coils, and an RF pulse applicator. The operation of the MR imaging apparatus is controlled by the controller.

The controller is a computer, processor, or other processing apparatus. The controller may be formed by several discrete processors; for example, the controller may comprise an MR imaging apparatus processor, which controls the MR imaging apparatus 110; an RT apparatus processor, which controls the operation of the RT apparatus; and a subject support surface processor which controls the operation and actuation of the subject support surface. The controller is communicatively coupled to a memory, e.g. a computer readable medium.

The linac device also comprises several other components and systems as will be understood by the skilled person. For example, in order to ensure the linac does not leak radiation, appropriate shielding is also provided.

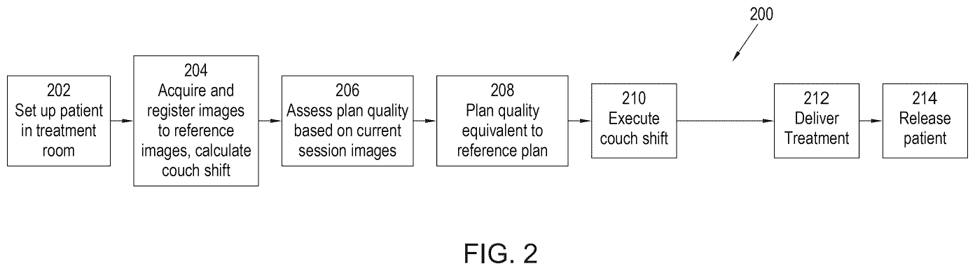

FIG. 2 shows a radiotherapy treatment workflow 200 according to one approach. The radiotherapy treatment workflow 200, which may also be referred to as a radiotherapy workflow, begins at block 202, in which a patient is set up in a treatment room. The patient set up comprises positioning the patient on an adjustable patient support of a radiotherapy device, such as those discussed in relation to FIG. 1. The patient may be positioned for treatment in a particular fraction session of their prescribed and/or scheduled radiotherapy treatment plan (including but not limited to the first fraction session). In other words, the patient may be positioned at a particular position according to the scheduled radiotherapy treatment plan and the radiation delivery apparatus may be positioned and/or prepared to deliver a prescribed dose according to the scheduled radiotherapy treatment plan.

The radiotherapy workflow 200 continues to block 204, in which at least one image of the patient positioned on the adjustable patient support is obtained. The image may be obtained using known patient imaging techniques, such as x-ray imaging, CBCT imaging, or/and MR imaging. The at least one image of the patient is compared with a reference image associated with the scheduled radiotherapy treatment plan. The reference image may have been obtained or generated and/or selected during the initial planning stage, for example. The comparison between the images may be in accordance with known techniques for image registration. The reference image indicates a reference position for a region of interest (ROI) of the patient. The region of interest may comprise a position of the target/tumour, and associated dimensions, as well as other anatomical features of the patient, such as the positions and associated dimensions of nearby organs, and in particular organs at risk. In many situations, a comparison of the images may indicate a difference or discrepancy between the position of the ROI in the obtained image of the patient on the adjustable patient support and the reference position for the ROI in the reference image associated with the scheduled radiotherapy plan. The discrepancy may be caused by, for example, inter-fractional changes in patient anatomy, such as swelling, fluid retention or other anatomical changes due to, for example, movements or different filling levels of internal organs. Weight gain or loss during therapy may also lead to a gradually growing discrepancy. Based on the comparison, a positional adjustment of the adjustable patient support is determined, which may be referred to as a “couch shift” in examples in which a patient couch is used as the adjustable patient support. The positional adjustment would move a current position of the ROI to a second position in which the ROI is more closely aligned with the reference position for the ROI. Such an adjustment is desirable in order to improve the accuracy and/or precision of delivery of radiation to the target.

At block 206, the scheduled radiotherapy treatment plan is assessed by determining whether delivering the scheduled radiotherapy treatment plan with the ROI at the second position would meet at least one pre-defined treatment plan quality criterion. Pre-defined treatment plan quality criteria may include a minimum prescribed dose for the target, and/or a maximum prescribed dose for at least one nearby organ at risk (OAR) as calculated by the dose planning system using Monte Carlo or another suitable high quality algorithm. For example, implementing a positional adjustment to compensate for a discrepancy in the position of the target on the day of treatment compared to within the scheduled treatment plan may mean that delivering radiation according to that plan, e.g. with particular multi-leaf collimator position or arrangement, may deliver an unacceptably high dose to a nearby OAR, which is problematic, and so the scheduled treatment plan would be determined not to meet a quality criterion related to the maximum dose to be delivered to that OAR. Accordingly, the determination at the block 206 may comprise computing or evaluating geometrical and/or anatomical positions for the proposed delivery with the ROI at the second position, along with dose depth characteristics. At block 208 of the radiotherapy workflow 200, it is determined whether the proposed delivery of the scheduled treatment plan with the ROI at the second position is equivalent to the reference plan in terms of the at least one treatment plan quality criterion. If so the workflow path executes 210 the positional adjustment to move the ROI to the second position, and delivering 214 treatment according to the originally scheduled radiotherapy treatment plan. The patient is then released at block 216, after which the workflow ends.

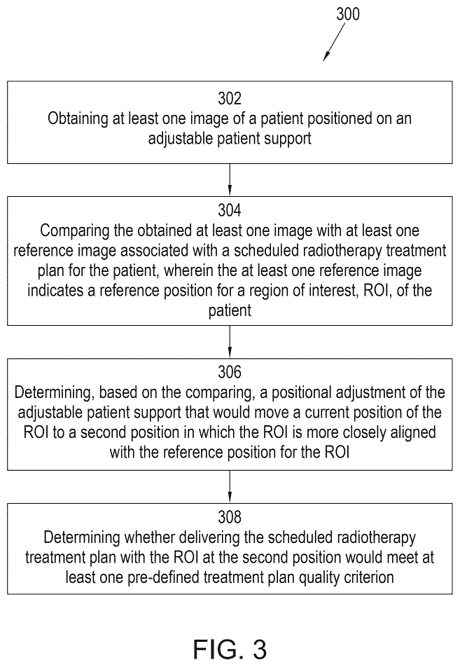

FIG. 3 shows a computer-implemented method 300. The method is suitable for being performed on a radiotherapy treatment day, when a patient is to have a fraction of radiation delivered to a region of interest according to their scheduled treatment plan. The radiotherapy workflow 200 of FIG. 2 is an exemplary implementation of method 300. Each of the blocks of the method 300 may be performed in accordance with a corresponding block(s) of the radiotherapy workflow 200 of FIG. 2, including optional exemplary implementations associated with a particular block. However, the method 300 is more general, and need not comprise all of the stages of the radiotherapy workflow 200 of FIG. 2.

The method 300 comprises, at block 302, obtaining at least one image of a patient positioned on an adjustable patient support. As described above in relation to FIG. 2, this image may be an MR image, a CT, CBCT image, or another medical imaging modality. At block 304, the method 300 comprises comparing the obtained at least one image with at least one reference image associated with a scheduled radiotherapy treatment plan for the patient, wherein the at least one reference image indicates a reference position for a region of interest, ROI, of the patient. The at least one reference image may be an image using during generation of the patient's radiotherapy treatment plan, for example. As described above, comparing may comprise registering the at least one image obtained at block 302 with the at least one reference image obtained at block 304.

At block 306, the method 300 comprises determining, based on the comparing, a positional adjustment of the adjustable patient support that would move a current position of the ROI to a second position in which the ROI is more closely aligned with the reference position for the ROI. For example, based on the registered images, it may be determined that there is a discrepancy or ‘offset’ between the position of an ROI in the reference image(s) and the position of the ROI in the newly obtained image(s). It may further be determined that this offset can be ‘corrected for’, or minimised, by re-positioning the patient using a positional adjustment to be enacted via the patent support surface.

At block 308, the method comprises determining whether delivering the scheduled radiotherapy treatment plan with the ROI at the second position would meet at least one pre-defined treatment plan quality criterion. In an example, the pre-defined treatment plan quality criterion is the prescribed dose to be delivered to the target during that fraction of treatment. In some examples, multiple pre-defined treatment plan quality criteria are used, which may include the prescribed dose to be delivered to the target (also known as the planning target volume, PTV, dose) as well as a maximum allowable dose for one or more nearby organs at risk.

As set out above, the at least one treatment plan quality criterion comprises one or more of a minimum target dose threshold and a maximum organ at risk dose threshold. In some examples, respective maximum organ at risk dose thresholds are used for multiple organs. In some examples, fewer parameters or variables are used for the treatment plan quality criterion than were used in developing the initial treatment plan, allowing the assessment of the plan to be performed quickly and efficiently. In some examples, calculating the adapted radiotherapy plan comprises calculating multi-leaf collimator component parameters to be used in delivering treatment, and/or other radiotherapy delivery variables, such as gantry parameters.

Each of the implementations of the method 300 may be performed in a radiotherapy fraction session, i.e. in a single session with a patient positioned on the adjustable patient support.

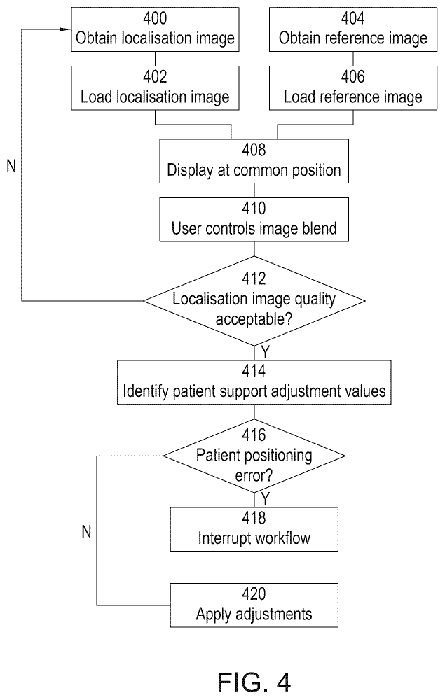

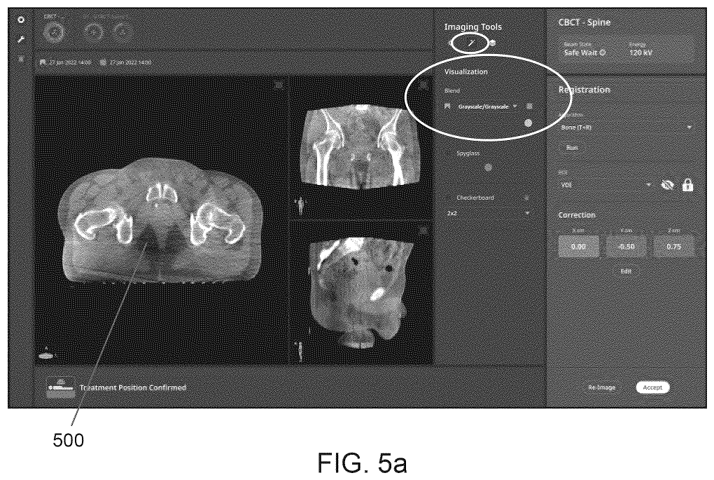

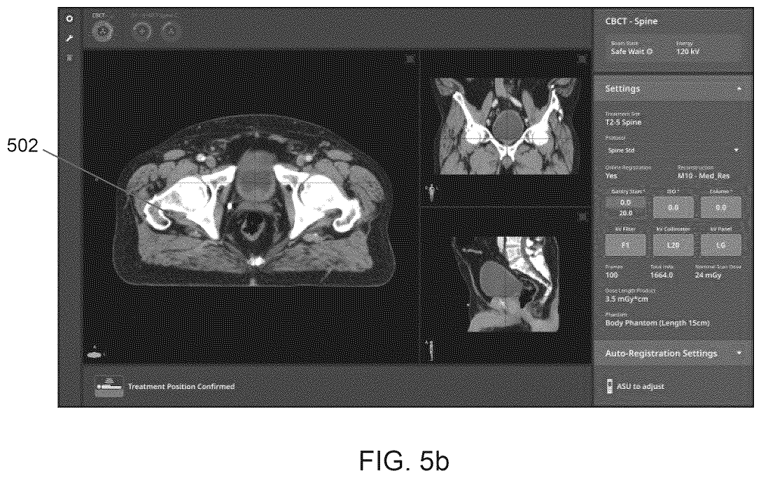

Referring now to FIG. 4, in conjunction with FIGS. 5-7, the steps involved in a method according to the a preferred embodiment can be further understood. At steps 400 and 404, a reference image and/or plan, and a localisation image of a patient positioned on an adjustable patient support are obtained and at steps 402 and 406 the images and plan are loaded for display for image quality review and comparison to identify patient support adjustment values and permit user image optimisation. FIG. 5a shows the localisation image at 500, and FIG. 5b shows the reference image at 502.

At step 408, the images are displayed at a common display location. The arrangement permits, subsequent to the image acquisition step, confirmation of the orientation of the displayed images to confirm a correct patient set up such that the orientation of the patient and the table matches the orientation of the reference plan image. By loading the images into the viewers in a fused display, later reload is not required.

At step 410 the blendable display is controllable between display of the localisation image only, display of the reference image only and display of a blend of localisation and reference image where the proportion of each image is controlled for example by a user slider or other interface control. In a preferred approach, the images are shown in greyscale at the most effective way for the user to observe any problems in the images such as motion artefact. As the user must validate that the localisation images are fit for use, the system can enforce initial localisation image review by initially defaulting the fused view to 100% localisation image.

At step 412, the localisation image can be checked for image quality and if the required images are deemed unacceptable, the user can choose to repeat image acquisition by selecting a “re-image” button or other user interface control and a renewed localisation image can be acquired by returning to step 400.

At step 414, the blended display can be used for comparison of the localisation and reference images to identify patient support adjustment values and permit user registration optimisation. This further permits comparing, and editing if needed, the position of the localisation images relative to the reference plan images to provide correction values needed to fine tune the table position. The manner in which the blended image is viewed can be user controllable and rely on display elements such as colouring or provision of a drop down menu, in which areas of alignment are displayed in an alternative colour to help determine a good match. Comparing the localisation and reference image to identify patient support adjustment values can comprise determining a positional adjustment of the adjustable patient support that would move a current position of an aspect of the localisation image to a position which the aspect is more closely aligned with a corresponding aspect of the reference image

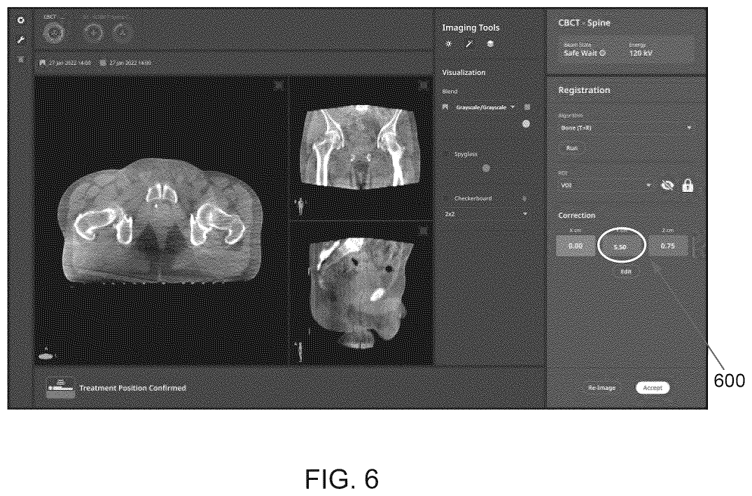

At step 416, a patient set up positioning error review can be performed for example by permitting the user to view correction values for the image at the same time that image quality is evaluated. Appropriate indicators, for example graphic user interface artefacts such as colour or other flags can inform the user that the patient support or couch is not in the position needed to correctly aim the treatment beam at the affected region. If the user observes large correction values indicating significant changes in support position, the user can elect to start once again as this can be an indication of a mistake when positioning the patient on the table. For example, a patient set up positioning error can be identified if the patient support adjustment values exceed a predetermined threshold. Referring to FIG. 6, it can be seen that the correction values 600 are flagged to indicate a potential workflow interrupt situation. In that case, the user can choose to interrupt the workflow at step 418. In a preferred approach, the user will then be presented with a list of possible patient set up return steps by the graphic user interface.

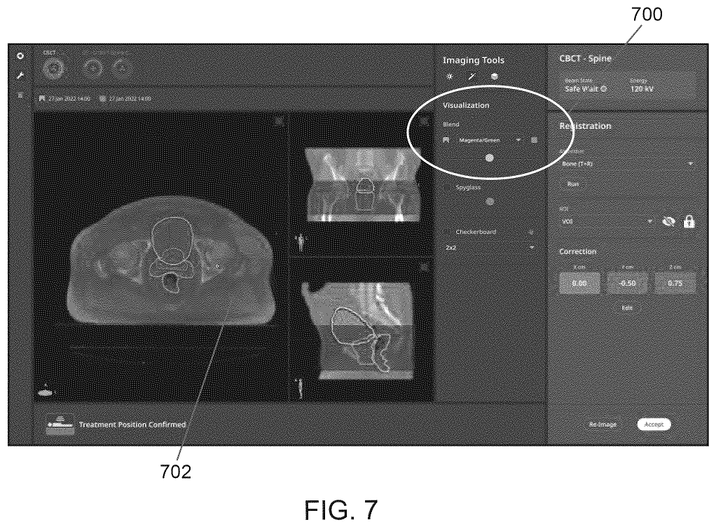

Once the user is satisfied with the registration, for example as shown at 700 in FIG. 7 indicating an acceptable match via colour and other visual indicators 702, at step 420 the user can accept the results, indicating acceptance of the acquired localisation of images as well as the image registration and prescribed correction values or table shifts. The correction values needed to fine tune the table position can be sent to the system responsible for moving the patient support so that the patient is aligned to the beam as intended by the reference plan. Upon acceptance, the table shifts are sent and the user is moved to the next activity for example where the table corrections are applied. The images and registration can be saved for offline review but it will be noted that in the preferred approach, registration edit tools are no longer enabled.

By ensuring the ordered steps in which reference and localisation images are loaded after 3D or 2D image acquisition, and in the preferred embodiment by defaulting the blending tools such that only localisation images are seen in an initial configuration, image quality review of the localisation image can be compulsory which reduces the chances of the user proceeding further in the workflow with images that are not fit for use. However, by virtue of the blendable view the user can additionally simultaneously view the offsets to determine if the patient set up is within reason, again accelerating and simplifying the processing required for the process. In particular, if neither reimaging nor patient repositioning is necessary then the user can use an appropriate interface control to update the viewers to show both the reference and localisation images to continue with image registration review without the requirement for additional loading steps.

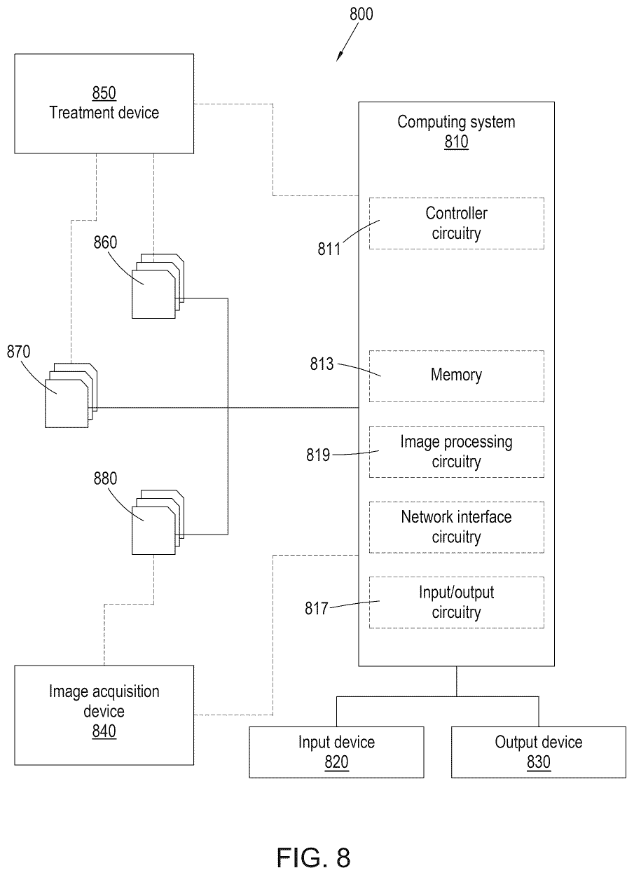

Also provided herein is a system for use in the radiotherapy workflow 200. The system is arranged to perform the methods disclosed herein, such as the methods 300 of FIG. 3, and comprises at least one processor and one or more non-transitory computer-readable media storing computer-executable instructions that, when executed by the one or more processors, cause the one or more processors to perform the methods disclosed herein. The system may comprise a radiotherapy device including radiotherapy delivery apparatus, such as those of FIGS. 1 and 8. Accordingly, the system may comprise a computing system, image acquisition device, treatment device, input device and/or output device like those of FIG. 8.

FIG. 8 illustrates a block diagram of one implementation of a radiotherapy system 800. The radiotherapy system 800 comprises a computing system 810 within which a set of instructions, for causing the computing system 810 to perform any one or more of the methods discussed herein, may be executed.

The computing system 810 shall be taken to include any number or collection of machines, e.g. computing device(s), that individually or jointly execute a set (or multiple sets) of instructions to perform any one or more of the methods discussed herein. That is, hardware and/or software may be provided in a single computing device, or distributed across a plurality of computing devices in the computing system. In some implementations, one or more elements of the computing system may be connected (e.g., networked) to other machines, for example in a Local Area Network (LAN), an intranet, an extranet, or the Internet. One or more elements of the computing system may operate in the capacity of a server or a client machine in a client-server network environment, or as a peer machine in a peer-to-peer (or distributed) network environment. One or more elements of the computing system may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine.

The computing system 810 includes controller circuitry 811 and a memory 813 (e.g., read-only memory (ROM), flash memory, dynamic random access memory (DRAM) such as synchronous DRAM (SDRAM) or Rambus DRAM (RDRAM), etc.). The memory 813 may comprise a static memory (e.g., flash memory, static random access memory (SRAM), etc.), and/or a secondary memory (e.g., a data storage device), which communicate with each other via a bus (not shown).

Controller circuitry 811 represents one or more general-purpose processors such as a microprocessor, central processing unit, accelerated processing units, or the like. More particularly, the controller circuitry 811 may comprise a complex instruction set computing (CISC) microprocessor, reduced instruction set computing (RISC) microprocessor, very long instruction word (VLIW) microprocessor, processor implementing other instruction sets, or processors implementing a combination of instruction sets. Controller circuitry 811 may also include one or more special-purpose processing devices such as an application specific integrated circuit (ASIC), a field programmable gate array (FPGA), a digital signal processor (DSP), network processor, or the like. One or more processors of the controller circuitry may have a multicore design. Controller circuitry 811 is configured to execute the processing logic for performing the operations and steps discussed herein.

The computing system 810 may further include a network interface circuitry 818. The computing system 810 may be communicatively coupled to an input device 820 and/or an output device 830, via input/output circuitry 817. In some implementations, the input device 820 and/or the output device 830 may be elements of the computing system 810. The input device 820 may include an alphanumeric input device (e.g., a keyboard or touchscreen), a cursor control device (e.g., a mouse or touchscreen), an audio device such as a microphone, and/or a haptic input device. The output device 830 may include an audio device such as a speaker, a video display unit (e.g., a liquid crystal display (LCD) or a cathode ray tube (CRT)), and/or a haptic output device. In some implementations, the input device 820 and the output device 830 may be provided as a single device, or as separate devices.

In some implementations, the computing system 810 may comprise image processing circuitry 819. Image processing circuitry 819 may be configured to process image data 880 (e.g. images, or imaging data), such as medical images obtained from one or more imaging data sources, a treatment device 850 and/or an image acquisition device 840. Image processing circuitry 819 may be configured to process, or pre-process, image data. For example, image processing circuitry 819 may convert received image data into a particular format, size, resolution or the like. In some implementations, image processing circuitry 819 may be combined with controller circuitry 811.

In some implementations, the radiotherapy system 800 may further comprise an image acquisition device 840 and/or a treatment device 850, such as those disclosed herein in the examples of FIGS. 1 to 3. The image acquisition device 840 and the treatment device 850 may be provided as a single device. In some implementations, treatment device 850 is configured to perform imaging, for example in addition to providing treatment and/or during treatment. The treatment device 850 comprises the main radiation delivery components of the radiotherapy system, such as the linac.

Image acquisition device 840 may be configured to perform positron emission tomography (PET), computed tomography (CT), magnetic resonance imaging (MRI), or other suitable imaging techniques.

Image acquisition device 840 may be configured to output image data 880, which may be accessed by computing system 810. Treatment device 850 may be configured to output treatment data 860, which may be accessed by computing system 810.

Computing system 810 may be configured to access or obtain treatment data 860, planning data 870 and/or image data 880. Treatment data 860 may be obtained from an internal data source (e.g. from memory 813) or from an external data source, such as treatment device 850 or an external database. Planning data 870 may be obtained from memory 813 and/or from an external source, such as a planning database. Planning data 870 may comprise information obtained from one or more of the image acquisition device 840 and the treatment device 850. Accordingly, computing system 810 is arranged to implement the methods disclosed herein relating to handling treatment plans and treatment quality criteria.

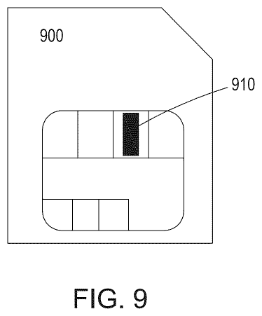

The various methods described above may be implemented by a computer program. The computer program may include computer code (e.g. instructions) 910 arranged to instruct a computer to perform the functions of one or more of the various methods described above. The steps of the methods described above may be performed in any suitable order. The computer program and/or the code 910 for performing such methods may be provided to an apparatus, such as a computer, on one or more computer readable media or, more generally, a computer program product 900)), depicted in FIG. 9. The computer readable media may be transitory or non-transitory. The one or more computer readable media 900 could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD. The instructions 910 may also reside, completely or at least partially, within the memory 813 and/or within the controller circuitry 811 during execution thereof by the computing system 810, the memory 813 and the controller circuitry 811 also constituting computer-readable storage media.

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may comprise a special-purpose processor, such as an FPGA or an ASIC. A hardware component may also include programmable logic or circuitry that is temporarily configured by software to perform certain operations.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing”, “enabling”, “maintaining”, “identifying”, “calculating”, “displaying”, or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A computer-implemented method for use in a radiotherapy workflow, comprising:
loading a reference image;
obtaining at least one localization image of a patient positioned on an adjustable patient support; and
loading the at least one localization image for display for user image quality review and for comparison with the reference image to identify patient support adjustment values and permit user image registration optimization;
wherein the at least one localization image and the reference image are loaded for display at a common display location, wherein the patient support adjustment values are displayed for patient setup positioning error review and upon receiving user input confirming the at least one localization image is of acceptable quality and image registration is optimized, and wherein the patient support adjustment values are sent for application to the adjustable patient support.

2. The computer-implemented method as claimed in claim 1, wherein the at least one localization image and the reference image are loaded for blendable display.

3. The computer-implemented method as claimed in claim 2, wherein the blendable display is controllable between display of localisation image only, display of reference image only and display of a blend of localisation and reference image.

4. The computer-implemented method as claimed in claim 3, wherein the blendable display is user controllable.

5. The computer-implemented method as claimed in claim 3, wherein, after the reference image and the at least one localization image are loaded, the blendable display defaults initially to display of the at least one localization image only.

6. The computer-implemented method as claimed in claim 1, wherein comparing the at least one localization image with the reference image to identify patient support adjustment values comprises determining a positional adjustment of the adjustable patient support to move a current position of an aspect of the at least one localization image to a position in which the aspect is more closely aligned with a corresponding aspect of the reference image.

7. The computer-implemented method as claimed in claim 1, wherein comparing the at least one localization image with the reference image to identify patient support adjustment values comprises identifying a patient setup positioning error and interrupting the radiotherapy workflow.

8. The computer-implemented method as claimed in claim 6, wherein a patient setup positioning error is identified when the patient support adjustment values exceed a predetermined threshold.

9. The computer-implemented method as claimed in claim 1, wherein the method is performed during a radiotherapy fraction session.

10. A system for use in a radiotherapy workflow, the system comprising:
at least one processor; and
one or more non-transitory computer-readable media storing computer-executable instructions that, when performed by the at least one processor, cause the at least one processor to perform a method comprising:
loading a reference image;
obtaining at least one localization image of a patient positioned on an adjustable patient support; and
loading the at least one localization image for display for user image quality review and for comparison with the reference image to identify patient support adjustment values and permit user image registration optimization;
wherein the at least one localization image and the reference image are loaded for display at a common display location, wherein the patient support adjustment values are displayed for patient setup positioning error review and upon receiving user input confirming the at least one localization image is of acceptable quality and image registration is optimized, the patient support adjustment values are sent for application to the adjustable patient support.

11. The system as claimed in claim 10, wherein the at least one localization image and the reference image are loaded for blendable display.

12. The system as claimed in claim 11, wherein the blendable display is controllable between display of at least one localization image only, display of the reference image only, and display of a blend of the at least one localization image and reference image.

13. The system as claimed in claim 12, wherein the blendable display is user controllable.

14. The system as claimed in claim 12, wherein, after the reference image and the at least one localization image are loaded, the blendable display defaults initially to display of the at least one localization image only.

15. The system as claimed in claim 10, wherein comparing the at least one localization image with the reference image to identify patient support adjustment values comprises identifying a patient setup positioning error and interrupting the radiotherapy workflow.

16. The system as claimed in claim 15, wherein a patient setup positioning error is identified when the patient support adjustment values exceed a predetermined threshold.

17. A non-transitory computer-readable medium containing instructions that, when performed by a processor, cause the processor to perform operations, the operations including:
loading a reference image;
obtaining at least one localization image of a patient positioned on an adjustable patient support; and
loading the at least one localization image for display for user image quality review and for comparison with the reference image to identify patient support adjustment values and permit user image registration optimization;
wherein the at least one localization image and the reference image are loaded for display at a common display location, wherein the patient support adjustment values are displayed for patient setup positioning error review and upon receiving user input confirming the at least one localization image is of acceptable quality and image registration is optimized, the patient support adjustment values are sent for application to the adjustable patient support.

18. The non-transitory computer-readable medium as claimed in claim 17, wherein the at least one localization image and the reference image are loaded for blendable display.

19. The non-transitory computer-readable medium as claimed in claim 18, wherein the blendable display is controllable between display of localisation image only, display of reference image only and display of a blend of localisation and reference image.

20. The non-transitory computer-readable medium as claimed in claim 19, wherein the blendable display is user controllable.

21. The non-transitory computer-readable medium as claimed in claim 18, wherein, after the reference image and at least one localization image are loaded, the blendable display defaults initially to display of the at least one localization image only.

22. The non-transitory computer-readable medium as claimed in claim 17, wherein comparing the at least one localization image with the reference image to identify patient support adjustment values comprises identifying a patient setup positioning error and interrupting a radiotherapy workflow.

23. The non-transitory computer-readable medium as claimed in claim 17, wherein a patient setup positioning error is identified when the patient support adjustment values exceed a predetermined threshold.
